(19) United States
(12) Patent Application Publication
(10) Pub. No.: US 2008/0188772 A1
(43) Pub. Date: Aug. 7, 2008

(54) Device for spatial localization of a movable part of the body

(75) Inventors: Thomas Broennimann, Wolfisberg (CH); Laurent Cloutot, Killwangen (CH)

Correspondence Address: SIEMENS SCHWEIZ AG;I-47, INTELLECTUAL PROPERTY, ALBISRIEDERSTRASSE 245, ZURICH, CH-8047, omitted

(73) Assignee: Siemens Schweiz AG, Zuerich (CH)

(21) Appl. No.: 12/012,712

(22) Filed: Feb. 5, 2008

(30) Foreign Application Priority Data
Feb. 6, 2007 (EP) 07002475.7

(51) Int. Cl. A61B 7/00

(52) U.S. Cl. 600/586

(57) ABSTRACT

A device for spatially localizing a movable part of a body, wherein movement of the body part is within a movement volume at surface parts and inside a living being, has a sound receiver device and a sound source. The sound receiver device is outside the living being and has spatially distinguishable sound receiver inputs located in an X-Y-Z coordinate system. The sound source is outside the living being, and configured to emit a first sound wave to propagate as far as the movement volume. The sound receiver device detects a second sound wave having a defined excitation wavelength or frequency, wherein the second sound wave is generated when the first sound wave causes a spectral exciter to oscillate. The sound receiver device is positioned such that the second sound wave encounters at least a maximum number of sound receiver inputs.

### CROSS-REFERENCE TO RELATED APPLICATIONS

The present application claims priority to European Patent Application No. 07002475.7, filed on Feb. 6, 2007, which is incorporated herein by reference.

### BACKGROUND OF THE INVENTION

The invention relates to a device for spatial localization of a movable part of a body, and further to a method of determining three-dimensional coordinates of a body part of a living being in real time relative to a known three-dimensional coordinate system.

Numerous methods or devices exist for radiation therapy of a tumor, for example, by means of Gamma or proton radiation, or for imaging of a tumor within the body of a being. These methods and devices aim to ensure a most accurate and reproducible three-dimensional localization of the tumor, or more generally of a movable part of the body. A few methods (e.g., “in-vivo” imaging methods that use radiography with x-rays) are however classified as damaging (“invasive”) methods and should therefore only be used in a restricted-manner. Even the few damaging “in-vivo” methods, such as, for example, an imaging method using magnetic resonance, in addition to their complexity, their expense and their costs, remain difficult to combine with and use at the same time as a radiation treatment. Other methods (“ex-vivo”) use (e.g., optically) detectable features/markings which can be attached to a patient and/or detected relative to a known coordinate system. However these methods lack accuracy and also reproducibility if physiological changes of the patient (e.g., gaining weight) or unavoidable (which are in the worst case unexpected) movements (e.g., caused by breathing) occur during treatments or if there is repositioning of the patient/parts of the body between treatments. Furthermore, methods and apparatuses for holding patients or parts of a body on a support table, for example, during radiation therapy, or devices for measuring breathing exist; however, on the one hand these solutions reduce the “comfort” of the patient, or do not allow a 100%-accurate spatial determination/localization of a tumor in a known coordinate system.

### SUMMARY OF THE INVENTION

The underlying object of the invention is to specify a device for reliable, spatial (i.e., one-, two- or three-dimensional) localization (i.e., also for precise, spatially-dynamic following) of a movable part of the body (tumor, carcinoma, etc.) of a living being (i.e., including a human patient).

Furthermore, a potential danger with stressful (=invasive) radiations of the patient is to be avoided. Especially, the device should be able during a Gamma radiation therapy of a patient e.g., for destruction of a tumor or of a carcinoma, where possible in real time, to localize the part of the body “in-vivo” via precise absolute coordinates in a known coordinate system, where possibly three-dimensionally.

Accordingly, one aspect involves a device for spatially localizing a movable part of a body, wherein movement of the body part is within a movement volume at surface parts and inside a living being, having a sound receiver device and a sound source. The sound receiver device is outside the living being and has spatially distinguishable sound receiver inputs located in an X-Y-Z coordinate system. The sound source is outside the living being, and configured to emit a first sound wave to propagate as far as the movement volume. The sound receiver device detects a second sound wave having a defined excitation wavelength or frequency, wherein the second sound wave is generated when the first sound wave causes a spectral exciter to oscillate. The sound receiver device is positioned such that the second sound wave encounters at least a maximum number of sound receiver inputs.

Another aspect involves a method of determining three-dimensional coordinates of a body part of a living being in real time relative to a known three-dimensional coordinate system. A sound receiver device is arranged outside the living being, wherein the sound receiver device has a plurality of spatially distinguishable sound receiver inputs, which are located in a predetermined coordinate system. A sound source is arranged outside the living being and configured to emit at least one first sound wave to propagate as far as a movement volume. The sound source is activated to emit the first sound wave. The sound receiver device detects a second sound wave having a defined excitation wavelength or frequency, wherein the second sound wave is generated when the first sound wave causes a spectral exciter to oscillate, the spectral exciter having a predetermined position relative to the part of the body, and wherein the sound receiver device is positioned such that the second sound wave encounters at least a maximum number of sound receiver inputs.

The more sound receiver inputs are to determine the second sound wave, the more accurately the part of the body is localized and the better are the measurement dynamics of the device for localization. If this is not the case, the sound receiver inputs are rearranged so that they can detect the second sound wave.

The variable number 1, 2, 3 of the sound receiver inputs on the one hand allows the one-, two- or three-dimensional localization of the exciter in an absolute, measurement based manner in the desired coordinate system. With larger numbers of sound receiver inputs and/or sound sources, the measurement localization is undertaken more accurately, with greater measurement dynamics and/or more quickly, where necessary.

Thus, at least sound-related measurement signals emanating from the part of the body are determined by the sound receiver device of which the delay times, frequencies, phases and/or propagation amplitudes vary at each sound receiver input of the sound receiver device depending on the distance between the part of the body and the sound receiver input. By determining characteristics of measuring signals an evaluation of the distances between each sound receiver input and the birthplace of the second sound wave is undertaken by the exciter. The fact that the relative locations/distances between the origin of the second sound wave and the sound receiver inputs have been made known, and the fact that both or all sound receiver inputs in a known, spatial coordinate system are known in an absolute way enables the precise position of the origin of the second sound wave in the absolute coordinate system to be calculated quickly and easily. In this way, the invention allows a unique one-, two- or three-dimensional localization of a part of the body movable within a movement volume (=origin of the second sound wave) as well as a high-speed tracking of the movement of the associated part of the body.

The fact that what is referred to as an actual isocenter to be determined is able to be moved with the movement of the part of the body (=of the exciter), means that an output signal of a sound receiver input changes or a number of output signals from the sound receiver inputs change simultaneously. The deviations of the output signal formed and determined by this thus allow a spatially dynamic tracking of the part of the body (=of the exciter) with high accuracy, which occurs relative to a reference point (e.g., a required isocenter of a radiation system). An attempt is subsequently made to permanently determine newly occurring deviations relative to a previously localized actual isocenter (and to compensate for them, if necessary, by a triggered positioning means and to keep the actual isocenter from the detected exciter permanently known as the next required isocenter), so that the part of the body always remains detectable with the sound receiver device.

Naturally, more than one sound source or more than two spatially separated sound receiver inputs can be used. The accuracy and/or the speed of the localization/following are increased in this way. In this way, possible sound-absorbing locations (especially within the body) can be covered better/more intensively with sound waves. The intensity of the sound waves is tailored in the interests of minimally invasive characteristics such that a sick patient is not disturbed.

The inventive device can additionally function in real time, so that in the known coordinate system absolute 3D-coordinates of a tumor containing the exciter (=part of the body) are issued, e.g., over 100 images per second. This measure is primarily dependent on the sound measurement technology used (as well as the reverse computation of the measurement data in the individual coordinate system). Should the picked up measurement signals be too weak or have signal-to-noise ratios which are too high, short-duration pulse signals can be created from the sound source and/or a number of temporally displaced measuring signals determined at a sound receiver input, their explicit measured values statistically added or averaged. Thus the noise values of the measured values picked up are also advantageously averaged, but at the cost of the pick-up speed. Unexpected or undesired sound-related measuring signals can be filtered out at the sound receiver input, e.g., through an electrical filter which is connected downstream from an electro-acoustic converter provided as a sound receiver input. Also avoided for collision, shadowing or interference reasons is a component (sound source, sound propagation paths or sound receiver input) of the inventive device standing in or along a/the beam path or paths (with, e.g., a Gamma radiation for radio-therapeutic purposes, e.g., for radiation of a tumor or a carcinoma). This means that the components of the inventive device are able to be positioned to the side of the Gamma radiation with the aid of mechanical means, such as switches or positioning modules, and where necessary can be moved synchronously with the radiation system, even taking into account that the Gamma beam path must change its direction/position for three-dimensional, spatial following of a tumor in real time.

The following explains in greater detail advantageous applications of the device. Especially for an application in which the part of the body includes unhealthy cells, for example, in the form of a tumor or of a carcinoma, of which the three-dimensional coordinates can be determined relative to a known three-dimensional coordinate system in real time. Furthermore the device can be used for an application for numerous parts of the body, e.g., if the unhealthy cells to be localized are located on the eyes, on the face, in the lungs or in the chest area of a human being. And finally, the three-dimensional coordinates of the localized part of the body can be issued to a drive controller of an installation for therapeutic radiation of the part of the body or for three-dimensional imaging of the body or transferred or used for supporting a therapeutic planning tool.

### BRIEF DESCRIPTION OF THE SEVERAL VIEWS OF THE DRAWINGS

The invention is explained below in an exemplary embodiment with reference to the drawing. In the drawings:

FIG. 1 shows a first inventive device,

FIG. 2 shows a second inventive device in a radio-therapeutic radiation system,

FIG. 3 shows the second inventive device in a patient bed,

FIG. 4 shows a third inventive device, and

FIG. 5 shows a fourth inventive device.

### DETAILED DESCRIPTION OF THE INVENTION

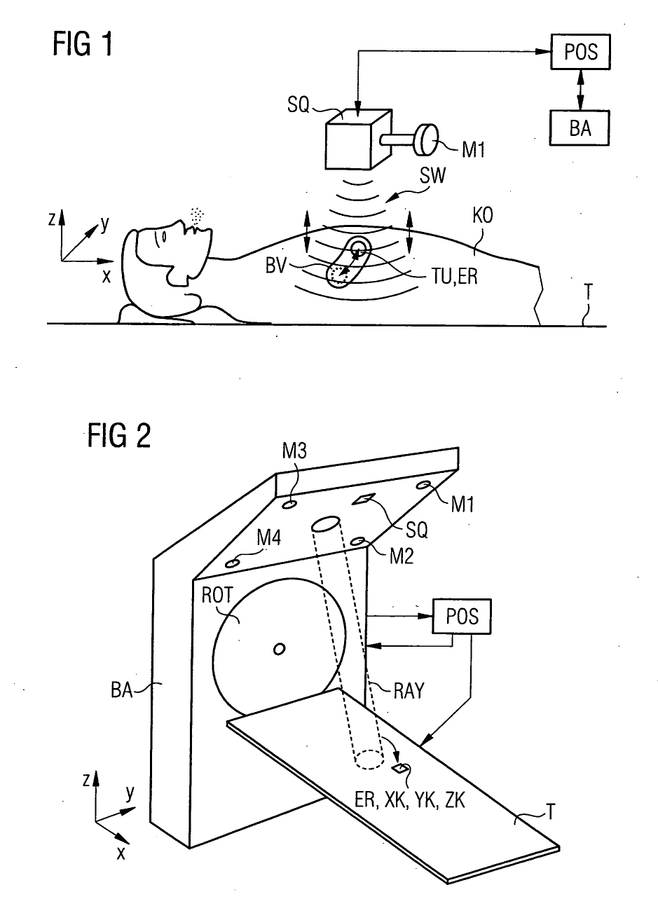

FIG. 1 shows a radiation system BA (only shown symbolically here) such as a linear accelerator for radio-therapeutic treatments, of which at least one beam output or axis is intended to target a tumor TU to be destroyed (here in the chest or lung area) of a person/patient KO lying on a table T. The breathing or the unexpected movement of the person inevitably causes the tumor TU to move relative to the pre-planned radiation target in the body KO and thus forms what is referred to as a movement volume BV which includes all positions of the tumor. Before a radiation therapy is performed it is usual to carry out a computer tomography in the chest or lung so that subsequently a repositioning of the inventive device is facilitated relative to the movement volume BV of the tumor determined thereby. After the computer tomography, the patient is positioned for regular radio-therapeutic treatments on the table T or another positionable table, if possible, in the same position as for the original computer tomography so that the tumor TU remains as the targeted isocenter of the radiation beam of the radiation system. However, the reproducibility of the regular positioning of the patient may not be reliable. In addition, breathing also complicates the localization of the tumor. Furthermore, the morphology of the patient can have changed within weeks. All these factors add up and lead to an imprecise localization of the targeted tumor.

A device to overcome these disadvantages is shown in FIG. 1 having a sound source SQ arranged externally to the patient which propagates at least one first sound wave SW as far as the tumor TU, such as with a known echographical method. Before the actual localization of the tumor, an exciter ER is present in the tumor which, on arrival of the first sound wave, emits a second sound-type wave at a frequency of the excitation wavelength depending on the exciter. Furthermore, the device has a microphone which forms a selective receiver of the second sound wave. The microphone M1 suitably isolates the sound wave coming back from the exciter from other sound waves in the vicinity of the tumor. After a suitable calibration of the delay times/amplitudes of the outgoing and returning sound waves it is possible to conduct an “echographical” distance measurement, which yields the distance between the wave source SQ (or of the microphone M1) and the exciter ER. Thus, a one-dimensional localization of the tumor in the coordinate system of the inventive device is first undertaken.

For a more precise, now bi-dimensional localization of the exciter or of the tumor, a further distance measurement must be undertaken according to the above principle. For this purposes, the sound receiver device (microphone M1) is expanded to act as a measurement device. On the one hand, a second measurement is undertaken with the same device, with the position of the sound source and/or of the microphone being changed spatially and in a known way. In general, the sound receiver device can feature at least two spatially separated sound receiver inputs which are positioned and oriented in a known coordinate system such that the second sound wave from the exciter arrives at both sound receiver inputs. This enables two distance measurements to be undertaken, which, when combined, make a two-dimensional localization of the tumor or of the exciter possible.

For three-dimensional localization of the exciter, the inventive device features in accordance with the previous exemplary embodiments three spatially separated sound receiver inputs, e.g., with three microphones, which are arranged outside the patient in a distributed manner, but are arranged close enough to the exciter so that the sound wave returning from the exciter is determined with an amplitude with sufficient signal-to-noise ratios at the microphones.

To form a number of sound receiver inputs, however, the previously described devices can only feature one electro-acoustic converter, preferably a microphone in accordance with FIG. 1. However, the number of sound receiver inputs can be varied in this case. For two- or three-dimensional localizations of the part of the body sought, the converter can be mounted on a hinged, rotatable and/or displaceable holder POS, or its location and orientation are able to be modulated to the movement volume by means of a switchover element. In this way, two, three or more chronologically consecutive measurements can be undertaken at the converter, with the spatial position of the converter being changed between the measurements or, formulated in more general terms, with the sound receiver input being displaced spatially. Such movements or switchover of the sound receiver input require all new positions of the sound receiver input in the coordinate system X, Y, Z of the table T to be known, using additional computing outlay where necessary.

In one embodiment, the exciter can have a specific sound-related resonance characteristic to enable the second sound wave to be created. Different types of exciters can be used in such cases. The exciter can, for example, be a substance injected into the body with known sound-related resonance characteristics, which resides exclusively and selectively in the area of the part of the body (=unhealthy cells of a tumor). Thus, cells arranged in the vicinity of the tumor cannot emit any second sound wave, so that only the tumor area is made to “resonate”. The exciter can, however, be a mechanical resonance element able to be implemented in the part of the body, and ideally in the form of an encapsulated, acoustic resonator. Such a resonance element can, for example, be placed in the tumor or at a relative position known to the tumor.

It is also conceivable to place a number of resonance elements in the tumor or in its vicinity, so that, as a result of the plurality of emitting resonance elements, an additive amplitude of the second sound waves to be determined thus becomes higher and easier to determine. This aspect also has the advantage of guaranteeing a more precise (since it is averaged out over a space) measurement of the second sound wave. On the other hand, the resonance elements can also emit different resonance frequencies so that measuring signals are better separated at the receiver device by spectral, selective filtering, for example. This aspect, depending on the range of the measurement, can avoid undesired interferences being formed between the emitted second sound waves.

It is conceivable to use as an exciter characteristic resonance features of a portion of the part of the body with a known, excitable resonant frequency. Also, a tumor cell, or more generally an especially diseased cell, can after investigation of its inherent resonance characteristic (by means, e.g., of a sound wave generator with variable frequency for the first sound wave, and by means of a spectrum analyzer for detection of returned second sound waves) have a different resonant frequency to that of healthy cells which are located around it. In this case, advantageously no injection of a substance, or no placing of a resonator element in the body has to be undertaken. This method is thus easier to bear for a patient under treatment, since it is non-invasive. In the case in which a resonator element is placed at the location of a tumor to be irradiated this element will also feature constructional materials which exhibit a functional resistance over a period of a number of weeks when a stress is imposed on them by a radio-therapeutic radiation.

It is also conceivable for the resonator element to simply dissolve over the course of time in the body since such an element consists of a very simple cavity which can be constructed in a material made from very different types (metal, plastic, etc). However, resonators with piezoelectric elements can also be used here which are very small and advantageously are electrically passive. This means than an exciter no longer has to be supplied with power by wiring as in the prior art, in which an induction coil implanted in a part of the body to be localized by means of external magnetic fields, with a wire for transmission of electrical signals between the part of the body and an electrical terminal outside the body is used. In the various embodiments described herein the resonator elements can be simply localized at the end of a patient treatment and removed, for example, with the aid of an endoscope if they are not soluble in the body. The size of resonator element is measured in millimeters (e.g., 0.5 to 2 mm).

To return to the formation of the sound receiver inputs, the sound receiver device can simply feature a number of spatially-separated electro-acoustic converters, preferably microphones. This is shown in FIG. 2 with four microphones M1, M2, M3, M4 in the radiation system BA being arranged around the ray output RAY for radio-therapeutic treatment. The extrinsic (=arranged externally from the patient) sound source SQ is likewise arranged between the microphones M1, M2, M3, M4, for example, next to the ray output RAY of the linear accelerator BA. It can also be seen that the sound source SQ intentionally has different distances to the microphones, so that all paths taken for sound waves from the “microphone sound source” pairs are unequal. This arrangement requires a pre-calibration of the different paths, however, it enables the device to be arranged to the side of the Gamma ray output RAY and installed simply in the radiation device BA. Shorter and higher paths of the sound waves are also formed in this way, which are better suited for a higher measurement dynamic, depending on the depth of the tumor within the body.

The sound receiver inputs can also be transparent to spectrally different sound wavelengths from the exciter. This requires the exciter to be capable of wideband emission or able to create different resonant frequencies. Clearly separated and thus interference-free sound signals are thus transferred to the respective microphones.

Likewise, in the interest of a sufficient measurement dynamic, the device has a sound regulator of the first sound wave connected to the sound source SQ with which an amplitude, a frequency and a phase of the first sound wave can be adjusted at the output of the sound source such that at least amplitude and/or phase signals of the second sound wave can be measured with a predefined signal-to-noise ratio at the sound receiver device (i.e., at all microphones if possible). The microphones can also be activated one after the other over time by different settings of the sound regulator or of the sound source being able to be retrieved. In this way, the measurement dynamic for each microphone is again optimized.

The sound receiver device (i.e., the microphone) can also feature acoustic filters for spectral isolation of the sound wavelength of the second sound wave. This prevents a measurement of one or more outside sound sources within as well as outside the body.

The sound receiver device is further connected to a processor unit in which by means of recorded data, preferably at the sound receiver device (=at the microphones) retrieved amplitude values of the second sound wave are able to be determined from the exciter, and by means of a detectable position of the sound receiver device relative to a known three-dimensional coordinate system X, Y, Z, three-dimensional coordinates XK, YK, ZK of the part of the body can be determined in the coordinate system (X, Y, Z) in real time. The detectable position of the sound receiver device can be determined by means of a measurement positioning module POS. The determined coordinates XK, YK, ZK for localization of the exciter in the coordinate system X, Y, Z can also be transferred to the positioning module POS which subsequently directs the radiation system BA and its ray onto the exciter. It is also possible for the positioning module POS to position the table T such that the exciter ER is held in the ray of the radiation system BA.

FIG. 2 shows an exemplary embodiment in which the inventive device has been integrated into the radiation system BA. However, if this integration is not possible (since, for example, the distance between the device and the patient is too great and the second sound wave would be too weak or the first sound wave were to have too high an amplitude), the device can likewise be arranged next to the radiation system BA and, for example, be accommodated with its own positioning means close to the body. The positioning means can feature any conceivable, mechanical holder. The main factor is that they deliver the location/orientation of the inventive device relative to the absolute coordinate system, so that the tumor can be localized in the same coordinate system and accordingly the radiation system BA, the table T or other medical components are repositioned in relation to the tumor (=exciter ER).

So that sufficient signal-to-noise ratios are ensured when picking up the second sound wave at the different microphones M1, M2, M3, M4, although the tumor can move and the attenuation of the sound wave varies, microphones can be arranged at different distances from the targeted part of the body. Thus, at least some of the microphones (which are close to the part of the body) are very well suited to signals with weak signal-to-noise ratios, and also some of the other microphones are suited to signals with high signal-to-noise ratios. The same then applies to the sound source SQ which can feature a number of sound sources arranged at different distances from the targeted part of the body.

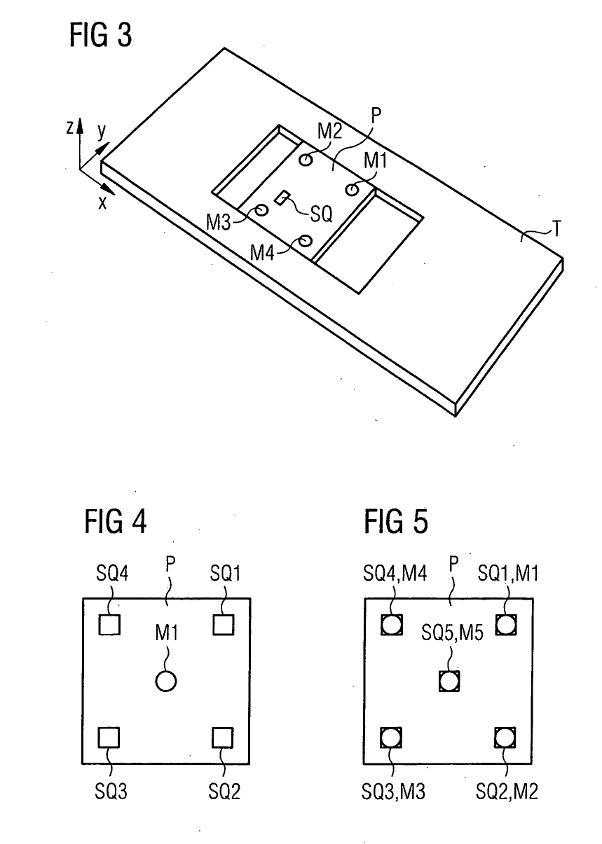

FIG. 3 shows a further exemplary embodiment with four microphones M1, M2, M3, M4 and a sound source SQ in accordance with FIG. 2, however, these components are arranged in a plate P which is anchored between the patient and the table T. This can be made possible, for example, by a recess in the table T extending lengthwise along the table in which the plate P is embedded and is able to be fixed at a location of the recess depending on where the exciter to be localized is located in the body. Thus, the sound source SQ emits the first sound source close to the skin on the back of the body of a recumbent patient and is still arranged to the side of a beam path (not shown here) of a linear accelerator. It is thus protected from possibly damaging radiation. The same then applies to the microphones. The plate P can, if necessary, be coupled to positioning means of its own, which allow movements of the plate relative to the table T or to the patient. This is however necessary if a too great a number of microphones could no longer detect the second sound wave (e.g., because the patient has moved a great deal).

FIG. 4 shows an exemplary embodiment which may be used individually or in combination with the previous exemplary embodiments in accordance with FIG. 2 (integrated into a radiation system) or FIG. 3 (on a plate P at table T). Here, instead of an individual sound source, the device features a number of sound sources SQ1, SQ2, SQ3, SQ4. This has the advantage that the exciter can be better irradiated with sound waves, especially with tumors which are located deep within the body. Alternatively, the four sound sources here can also have different radiation characteristics, e.g., in the interests of more comprehensive adjustment options of amplitudes, wavelengths, etc of the generated sound waves or in the interests of a more comprehensive or more precise resonance-specific adaptation to the exciter or to a number of exciters (if necessary, with different resonance characteristics). Here, only one microphone M1 is shown and is arranged centrally between the sound sources. This means however that the incident radio-therapeutic ray in accordance with FIG. 2 should be directed next to the microphone M1 to avoid interfering with measurements made by the microphone M1.

FIG. 5 now shows an exemplary embodiment in accordance with FIG. 4, in which five microphones M1, M2, M3, M4, M5 are combined with five sound sources SQ1, SQ2, SQ3, SQ4, SQ5 distributed over the plate P. This device thus gives a high level of flexibility because of a diversity of settings for sound-related characteristics of each component (=each microphone and each sound source) able to be emitted as well as received as well as through the large number of the components greater and more accurate measurement characteristics for localization of a an exciter. If necessary, one of the sound sources and one of the microphones can be realized as an individual component, e.g., by an oscillation element as per the model of a loudspeaker.

In FIGS. 3, 4 and 5, the arrangement of the sound sources and the microphones in the flat plate is further selected such that for a radiation system BA with an axis of rotation ROT in relation to the table T, the ray which is thus able to be rotated around the table T remains out of contact with the sound sources and the microphones, so that the measurements made by the components of the inventive device are not disturbed and thus so that the desired localization/following of the exciter/part of the body is always performed reliably.

What is claimed is:

1. A device for spatially localizing a movable part of a body, wherein movement of the body part is within a movement volume at surface parts and inside a living being, comprising:
a sound receiver device arranged outside the living being and having a plurality of spatially distinguishable sound receiver inputs, which are located in a predetermined coordinate system; and
a sound source arranged outside the living being, and configured to emit at least one first sound wave to propagate as far as the movement volume,
wherein the sound receiver device is configured to detect a second sound wave having a defined excitation wavelength or frequency,
wherein the second sound wave is generated when the first sound wave causes a spectral exciter to oscillate, the spectral exciter having a predetermined position relative to the part of the body, and
wherein the sound receiver device is positioned such that the second sound wave encounters at least a maximum number of sound receiver inputs.

2. The device of claim 1, wherein the sound receiver device has an electro-acoustic converter mounted on at least one of a hinged, rotatable and displaceable holder.

3. The device of claim 1, wherein the sound receiver device has an electro-acoustic converter or of which the location and orientation relative to the movement volume is able to be varied by means of a switchover element or forming a number of sound receiver inputs the sound receiver device features an

4. The device of claim 1, wherein the sound receiver device has a number of spatially separated electro-acoustic converters to form a number of sound receiver inputs.

5. The device of claim 4, wherein the sound receiver inputs are transparent for spectrally different sound wavelengths.

6. The device of claim 1, further comprising a sound regulator for the first sound wave that is connected to the sound source, wherein the sound regulator is configured to adjust an amplitude, a frequency and a phase of the first sound wave at an output of the sound source such that at least one of amplitude and phase signals of the second sound wave are measured at a predefined signal-to-noise ratio at the sound receiver device.

7. The device of claim 1, wherein the sound receiver device includes acoustic filters for spectral isolation of the sound wavelength of the second sound wave.

8. The device of claim 1, further comprising a processor unit connected to the sound receiver device in which by means of recorded data, preferably amplitude values of the second sound wave obtained at the sound receiver device and by means of detectable position of the sound receiver device relative to a known three-dimensional coordinate system three-dimensional coordinates of the part of the body are able to be determined in the coordinate system in real time.

9. The device of claim 1, wherein the exciter is a proportion of the part of the body with a known, excitable resonant frequency.

10. The device of claim 9, wherein the exciter is a substance able to be injected into the body with known sound-related resonance characteristics which resides exclusively and selectively in the area of the part of the body.

11. The device of claim 9, wherein the exciter comprises a mechanical resonance element able to be implemented in the part of the body, ideally in the form of an encapsulated, acoustic resonator.

12. The device of claim 10, wherein the resonator element has constructional materials which exhibit a mechanical functional resistance lasting over a number of weeks to a stress imposed by a radio-therapeutic radiation on the resonator element.

13. The device of claim 1, wherein the sound source and the sound receiver device are arranged to a side of radio-therapeutic radiation.

14. The device of claim 1, wherein the sound source and the sound receiver device are arranged in a plate.

15. The device of claim 14, wherein the plate is arranged between the being and a table on which the being lies.

16. A method of determining three-dimensional coordinates of a body part of a living being in real time relative to a known three-dimensional coordinate system, comprising:
arranging a sound receiver device outside the living being, wherein the sound receiver device has a plurality of spatially distinguishable sound receiver inputs, which are located in a predetermined coordinate system;
arranging a sound source outside the living being, wherein the sound source is configured to emit at least one first sound wave to propagate as far as a movement volume;
activating the sound source to emit the first sound wave; and
detecting via the sound receiver device a second sound wave having a defined excitation wavelength or frequency, wherein the second sound wave is generated when the first sound wave causes a spectral exciter to oscillate, the spectral exciter having a predetermined position relative to the part of the body, and wherein the sound receiver device is positioned such that the second sound wave encounters at least a maximum number of sound receiver inputs.

17. The method of claim 16, further comprising outputting the three-dimensional coordinates of the part of the body to a controller of an installation for therapeutic radiation of the part of the body.
